(19) United States
(12) Patent Application Publication
(10) Pub. No.: US 2004/0249242 A1
(43) Pub. Date: Dec. 9, 2004

(54) Multi-panel cardiac harness

(76) Inventors: Lilip Lau, Los Altos, CA (US); James Hong, Palo Alto, CA (US); Steven Meyer, Oakland, CA (US); Matthew G. Fishler, Sunnyvale, CA (US); Craig Mar, Fremont, CA (US)

Correspondence Address: FULWIDER PATTON LEE & UTECHT, LLP, HOWARD HUGHES CENTER, 6060 CENTER DRIVE, TENTH FLOOR, LOS ANGELES, CA, 90045, US

(21) Appl. No.: 10/811,245

(22) Filed: Mar. 25, 2004

Related U.S. Application Data
(60) Provisional application No. 60/458,991, filed on Mar. 28, 2003.

(51) Int. Cl. A61F 2/00; A61B 19/00; B21F 35/00; B44C 1/22

(52) U.S. Cl. 600/37; 29/896.92; 216/48

(57) ABSTRACT

A cardiac harness configured to be fit around at least a portion of a patient's heart, including a conductive material that is coated with a dielectric coating to electrically insulate at least the heart tissue from the conductive material. The cardiac harness applies a compressive force on the heart during diastole and systole. The cardiac harness includes an arrangement that provides no electrical continuity circumferentially about the harness, so that if an electric current created by a defibrillation device is applied to a patient who has a harness that is placed on their heart, the electric current will pass through the heart unimpeded instead of being conducted around the heart through the harness.

### CROSS-REFERENCE TO RELATED APPLICATION

This application depends for priority upon U.S. Provisional Patent Application No. 60/458,991, filed Mar. 28, 2003, which is herein incorporated by reference in its entirety.

### BACKGROUND OF THE INVENTION

The present invention relates to a device for treating heart failure. More specifically, the invention relates to a cardiac harness configured to be fit around at least a portion of a patient's heart. The cardiac harness includes an arrangement that provides no electrical continuity circumferentially about the harness, and therefore allows an electric current to pass through the heart unimpeded. In a situation where a defibrillating electrode positioned inside the right ventricle of a patient is connected to an implantable cardiac defibrillator (“ICD”), the arrangement of the cardiac harness will allow the defibrillating current to pass from the electrode inside of the heart to the ICD without the current being conducted around the heart through the harness. Also, if defibrillator paddles are applied to a harness that is placed on a patient's heart, the electric current created between the paddles would pass through the heart instead of being conducted around the heart through the harness.

Congestive heart failure (“CHF”) is characterized by the failure of the heart to pump blood at sufficient flow rates to meet the metabolic demand of tissues, especially the demand for oxygen. One characteristic of CHF is remodeling of at least portions of a patient's heart. Remodeling involves physical change to the size, shape and thickness of the heart wall. For example, a damaged left ventricle may have some localized thinning and stretching of a portion of the myocardium. The thinned portion of the myocardium often is functionally impaired, and other portions of the myocardium attempt to compensate. As a result, the other portions of the myocardium may expand so that the stroke volume of the ventricle is maintained notwithstanding the impaired zone of the myocardium. Such expansion may cause the left ventricle to assume a somewhat spherical shape.

Cardiac remodeling often subjects the heart wall to increased wall tension or stress, which further impairs the heart's functional performance. Often, the heart wall will dilate further in order to compensate for the impairment caused by such increased stress. Thus, a cycle can result, in which dilation leads to further dilation and greater functional impairment.

Historically, congestive heart failure has been managed with a variety of drugs. Devices have also been used to improve cardiac output. For example, left ventricular assist pumps help the heart to pump blood. Multi-chamber pacing has also been employed to optimally synchronize the beating of the heart chambers to improve cardiac output. Various skeletal muscles, such as the latissimus dorsi, have been used to assist ventricular pumping. Researchers and cardiac surgeons have also experimented with prosthetic “girdles” disposed around the heart. One such design is a prosthetic “sock” or “jacket” that is wrapped around the heart.

Patients suffering from congestive heart failure often are at risk to additional cardiac failures, including cardiac arrhythmias. This irregularity in heartbeat is caused by irregularities in the electrical conduction system of the heart. For example, damage from a cardiac infarction can interrupt the electrical signaling of the heart. In some instances, implantable devices, such as pacemakers, help to regulate cardiac rhythm and stimulate heart pumping. A problem with the heart's electrical system can sometimes cause the heart to fibrillate. During fibrillation, the heart does not beat normally, and sometimes does not pump adequately. A cardiac defibrillator can be used to restore the heart to normal beating. One type of defibrillator includes a pair of electrode paddles applied to the patient's chest. The defibrillator generates an electric field between electrodes. An electric current passes through the patient's heart and stimulates the heart's electrical system to help restore the heart to regular pumping. Other types of defibrillators that function similarly include automatic external defibrillators, which are known in the art.

Sometimes a patient's heart begins fibrillating during heart surgery or other open-chest surgeries. In such instances, a special type of defibrillating device is used. An open-chest defibrillator includes special electrode paddles that are configured to be applied to the heart on opposite sides of the heart. A strong electric field is created between the paddles, and an electric current passes through the heart to defibrillate the heart and restore the heart to regular pumping.

In some patients that are especially vulnerable to fibrillation, an implantable heart defibrillation device may be used. Such an implantable device generally includes two or more electrodes mounted directly on or adjacent the heart wall. If the patient's heart begins fibrillating, these heart-mounted electrodes will generate an electric field there between in a similar manner as the other defibrillators discussed above. Other implantable devices include a power source, such as an ICD or an active can, that is connected to a transvenous electrode positioned inside the right ventricle of the heart. This type of device generates an electric field between the electrode and the power source positioned near the heart to provide a shock to the heart.

Testing has indicated that when defibrillating electrodes are applied to a heart that is surrounded by a device made of electrically conductive material, much of the electrical current disbursed by the paddles is conducted around the heart by the conductive material, rather than through the heart. Thus, the efficacy of defibrillation is reduced. Further, testing also has shown that the electrically conductive device surrounding the heart can develop into a Faraday cage when a defibrillating current is applied to the heart.

Accordingly, the present invention includes several embodiments of a cardiac harness that enables defibrillation of the heart with the application of defibrillating paddles.

### SUMMARY OF THE INVENTION

In accordance with the present invention, a cardiac harness is configured to fit at least a portion of a patient's heart and includes a conductive material that is coated with a dielectric coating to electrically insulate the conductive material from an electric current that may be applied to the heart. In one embodiment, the conductive material is entirely coated with the dielectric coating so that the entire harness is electrically insulated. The conductive material can be a metallic wire, and preferably one having a shape memory property, and the dielectric coating can include Parylene™, silicone rubber, urethane, polytetrafluoroethylene, or an elastomer. In certain embodiments, the conductive material comprises a wire that formed into a plurality of hinge members.

In another embodiment, a cardiac harness which circumferentially surrounds a patient's heart and extends longitudinally from an apex portion to a base portion of the heart, includes a first portion and a second portion. The first portion is configured to be disposed closer to the apex portion of the heart than the second portion, and the first portion includes a plurality of interconnected panels that are electrically insulated from one another along respective longitudinal sides to inhibit electrical conduction circumferentially about the harness. In this embodiment, the second portion is electrically insulated from the first portion, and includes a plurality of circumferentially extending rings with a plurality of interconnected spring elements, where the rings are also electrically insulated from one another. The first and second portions may be coated with a dielectric material, and at least one non-conductive connector including the dielectric material connects the first portion to the second portion of the harness.

In yet another embodiment, a cardiac harness includes a first spring array and a second spring array, with each spring array including a plurality of zig portions interconnected with a plurality of zag portions such that the array is generally zigzag shaped. In this embodiment, the zig portions and zag portions include a plurality of interconnected spring elements. The first and second spring arrays are connected to one another at a plurality of discrete locations corresponding to interconnections of the zig portion with the zag portion. In one embodiment, the cardiac harness also includes an elongate coil, where one or more windings of the coil surround portions of the adjacent spring arrays at some of the discrete locations.

In another embodiment, a cardiac harness includes a plurality of interconnected spring members comprised of a conductive material. At least some of the spring members are connected to other spring members by a dielectric material, such that the spring members are substantially electrically insulated from each other.

Also in accordance with the present invention, a method of manufacturing a cardiac harness includes providing a metallic wire, covering the wire with a dielectric material, and forming the wire into a plurality of spring members. In one embodiment, the dielectric material is in the form of a tube that is slid over the metallic wire, such that the wire is insulated by the dielectric material. Any excess dielectric material is removed from the wire so that the shape of the dielectric material generally follows the shape of the spring members.

In another embodiment, a method of manufacturing a cardiac harness includes, etching at least one spring member out of a flat sheet of conductive material. In this embodiment the etched spring member is then coated with a dielectric material, such that the etched spring member is insulated by the dielectric material, and any excess dielectric material is removed from the etched spring member so that the shape of the dielectric material generally follows the shape of the spring members.

In each of the above embodiments of the cardiac harness, the dielectric coating is configured to prevent an electric field applied by any source from being conducted circumferentially around the heart by the metallic harness. Thus, if a defibrillating current is created between defibrillator paddles or electrodes position inside, on, or near the patient's heart, the defibrillating current will not be conducted around the heart through the harness, but instead, pass through the heart. As such, the effectiveness of the defibrillating shock is not defeated by the presence of the harness.

### BRIEF DESCRIPTION OF THE DRAWINGS

FIG. 1 depicts a schematic view of a heart with a prior art cardiac harness placed thereon.

FIGS. 2A-2B depict a spring hinge of a prior art cardiac harness in a relaxed position and under tension.

FIG. 3 depicts a prior art cardiac harness that has been cut out of a flat sheet of material.

FIG. 4 depicts the prior art cardiac harness of FIG. 3 formed into a shape configured to fit about a heart.

FIG. 5 depicts a perspective view of one embodiment of a cardiac harness having spring arrays connected together with a dielectric material.

FIG. 5A depicts a portion of one embodiment of a spring array emphasizing a zigzag configuration.

FIG. 5B depicts a portion of one embodiment of a spring array having a zigzag configuration.

FIG. 6 depicts one embodiment of a spring array having a decreasing number of spring members in successive rows from a base end to an apex end.

FIG. 7 depicts a perspective view of one embodiment of a cardiac harness having spring arrays attached to adjacent spring arrays by elongate coils.

FIG. 8 depicts a perspective view of another embodiment of a cardiac harness having spring arrays attached to adjacent spring arrays by a plurality of nonconductive connectors.

FIG. 9 depicts a side elevational view of yet another embodiment of a cardiac harness having a first portion and a second portion.

FIG. 9A depicts a top plan view of the cardiac harness shown in FIG. 9.

FIG. 9B depicts a side elevational view of the second portion of the cardiac harness shown in FIG. 9.

FIG. 9C depicts a side elevational view of the first portion of the cardiac harness shown in FIG. 9.

FIG. 9D depicts a side elevational view of the cardiac harness shown in FIG. 9, showing the first portion attached to the second portion by a nonconductive connector.

FIG. 9E depicts an embodiment of a cardiac harness having partial strands attached to the second portion of the harness, wherein one of the partial strands and two of the spring arrays are not covered with a dielectric material, but are electrically connected to a controller.

FIG. 10 depicts a perspective view of another embodiment of a cardiac harness having a plurality of rings covered with a dielectric material and disposed longitudinally adjacent to one another.

FIG. 10A depicts a partial cross-sectional view of opposite ends of each ring attached to one another by a connective junction.

FIG. 11 depicts an unattached elongated strand or series of spring elements.

FIG. 12 depicts a perspective view of another embodiment of a cardiac harness having a plurality of rings covered with a dielectric material and adjacent rings are interconnected with nonconductive connectors.

FIG. 12A depicts a connective junction joining opposite ends of a ring with a dielectric material.

FIG. 13A depicts a spring array having a harness portion and a leader portion.

FIG. 13B depicts an elongated strand having a harness portion and a leader portion.

FIGS. 14A-14B depict a schematic view of a wire being covered with a silicone tube.

### DETAILED DESCRIPTION OF THE PREFERRED EMBODIMENTS

This application relates to a method and apparatus for treating heart failure. As discussed in Applicants' co-pending application entitled “Expandable Cardiac Harness For Treating Congestive Heart Failure”, Ser. No. 09/634,043, which was filed on Aug. 8, 2000, the entirety of which is hereby expressly incorporated by reference herein, it is anticipated that remodeling of a diseased heart can be resisted or even reversed by alleviating the wall stresses in such a heart. The present application discusses certain embodiments and methods for supporting the cardiac wall. Additional embodiments and aspects are also discussed in Applicants' co-pending applications entitled “Device for Treating Heart Failure,” Ser. No. 10/242,016, filed Sep. 10, 2002, “Heart Failure Treatment Device and Method”, Ser. No. 10/287,723, filed Oct. 31, 2002, “Method and Apparatus for Supporting a Heart”, Ser. No. 10/338,934, filed Jan. 7, 2003, and “Method and Apparatus for Treating Heart Failure,” Ser. No. 60/409,113, filed Sep. 5, 2002, the entirety of each of which are hereby expressly incorporated by reference.

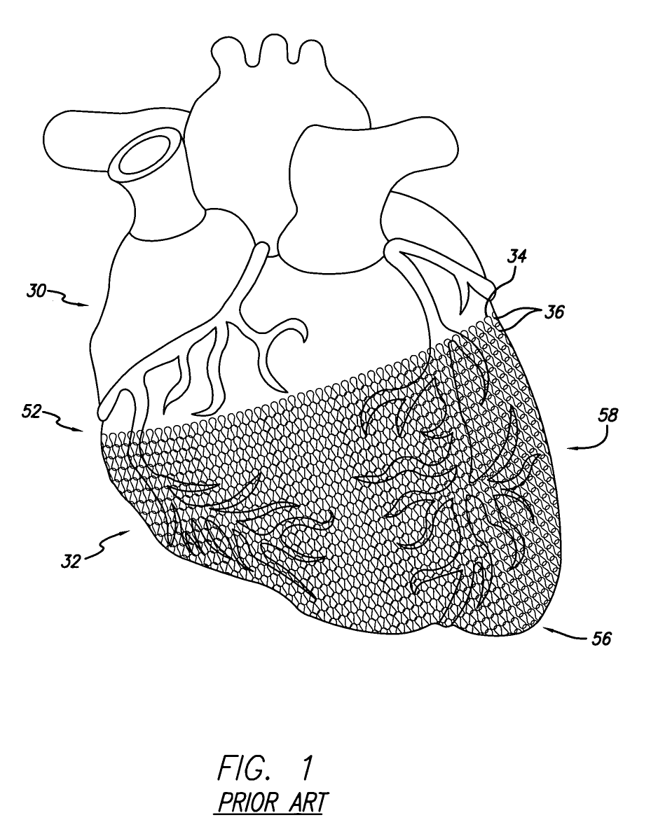

FIG. 1 illustrates a mammalian heart 30 having a prior art cardiac wall stress reduction device in the form of a harness 32 applied to it. The cardiac harness has a series of hinges or spring elements 34 that circumscribe the heart and, collectively, apply a mild compressive force on the heart so as to alleviate wall stresses.

The term “cardiac harness” as used herein is a broad term that refers to a device fit onto a patient's heart to apply a compressive force on the heart during at least a portion of the cardiac cycle. Other devices that are intended to be fit onto a heart and are referred to in the art as “girdles,” “socks,” “jackets,” or the like are included within the meaning of “cardiac harness.”

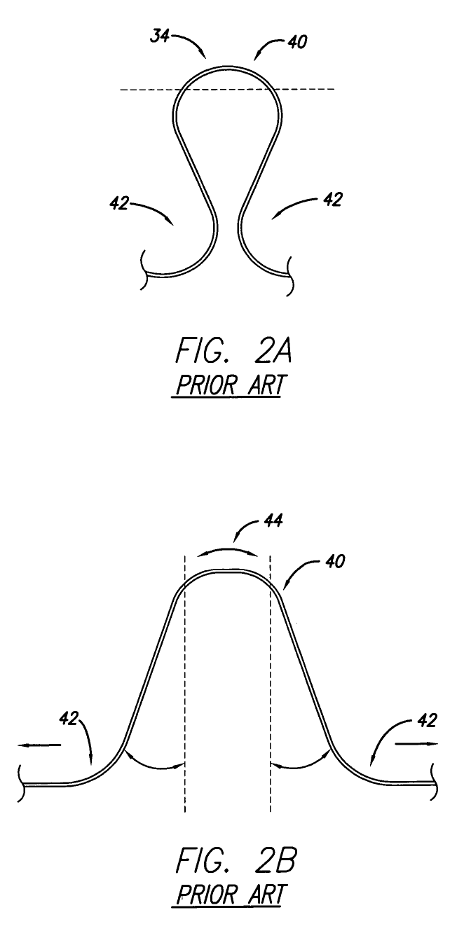

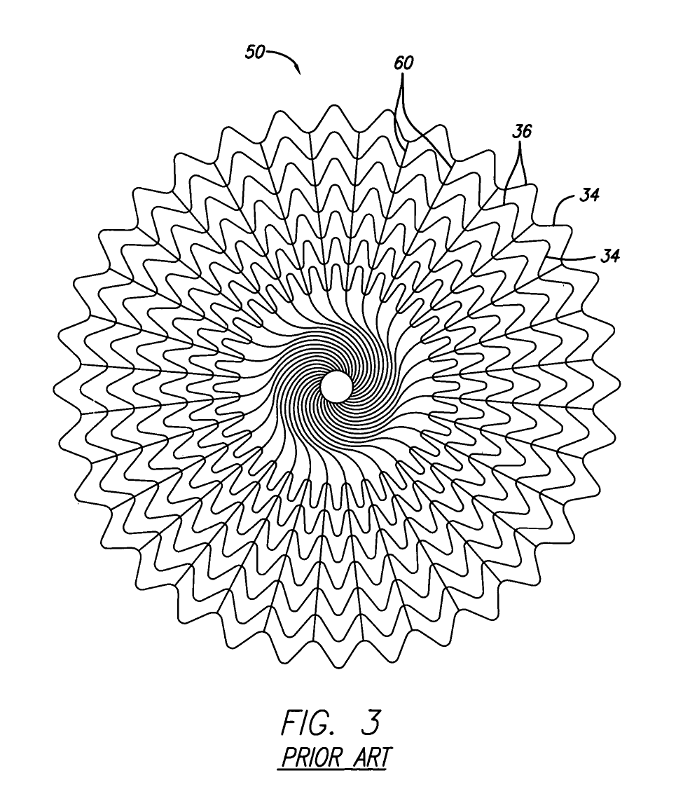

The cardiac harness 32 illustrated in FIG. 1 has at least one undulating strand 36 having a series of spring elements 34 referred to as hinges or spring hinges that are configured to deform as the heart 30 expands during filling. Each hinge provides substantially unidirectional elasticity, in that it acts in one direction and does not provide much elasticity in the direction perpendicular to that direction. For example, FIG. 2A shows one embodiment of a prior art hinge member at rest. The hinge member has a central portion 40 and a pair of arms 42. As the arms are pulled, as shown in FIG. 2B, a bending moment 44 is imposed on the central portion. The bending moment urges the hinge member back to its relaxed condition. Note that a typical strand comprises a series of such hinges, and that the hinges are adapted to elastically expand and retract in the direction of the strand.

In the harness illustrated in FIG. 1, the strands 36 of spring elements 34 are constructed of extruded wire that is deformed to form the spring elements.

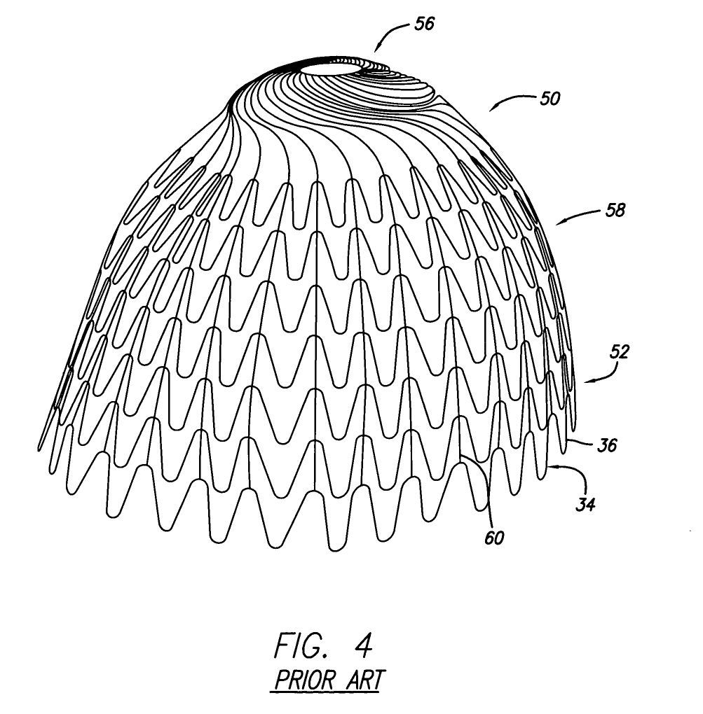

FIGS. 3 and 4 illustrate another prior art cardiac harness 50, shown at two points during manufacture of such a harness. The harness is first formed from a relatively thin, flat sheet of material. Any method can be used to form the harness from the flat sheet. For example, in one embodiment, the harness is photochemically etched from the material; in another embodiment, the harness is laser-cut from the thin sheet of material. The harness shown in FIGS. 3 and 4 has been etched from a thin sheet of Nitinol, which is a superelastic material that also exhibits shape memory properties. The flat sheet of material is draped over a form, die or the like, and is formed to generally take on the shape of at least a portion of a heart.

With further reference to FIGS. 1 and 4, the cardiac harnesses 32, 50 have a base portion 52, which is sized and configured to generally engage and fit onto a base region of a patient's heart; an apex portion 56, which is sized and shaped so as to generally engage and fit on an apex region of a patient's heart; and a medial portion 58 between the base and apex portions.

In the harness shown in FIGS. 3 and 4, the harness 50 has strands or rows 36 of undulating wire. As discussed above, the undulations have hinges/spring elements 34 which are elastically bendable in a desired direction. Some of the strands are connected to each other by interconnecting elements 60. The interconnecting elements help maintain the position of the strands relative to one another. Preferably the interconnecting elements allow some relative movement between adjacent strands.

The undulating spring elements 34 exert a force in resistance to expansion of the heart 30. Collectively, the force exerted by the spring elements tends toward compressing the heart, thus alleviating wall stresses in the heart as the heart expands. Accordingly, the harness helps to decrease the workload of the heart, enabling the heart to more effectively pump blood through the patient's body and enabling the heart an opportunity to heal itself. It should be understood that several arrangements and configurations of spring members can be used to create a mildly compressive force on the heart to reduce wall stresses. For example, spring members can be disposed over only a portion of the circumference of the heart or the spring members can cover a substantial portion of the heart.

As the heart expands and contracts during diastole and systole, the contractile cells of the myocardium expand and contract. In a diseased heart, the myocardium may expand such that the cells are distressed and lose at least some contractility. Distressed cells are less able to deal with the stresses of expansion and contraction. As such, the effectiveness of heart pumping reduces. Each spring member of the above cardiac harness is configured so that as the heart expands during diastole the spring members correspondingly will expand, thus storing expansion forces as bending energy in the spring. As such, the stress load on the myocardium is partially relieved by the harness. This reduction in stress helps the myocardium cells to remain healthy and/or regain health. As the heart contracts during systole, the disclosed prior art cardiac harness applies a moderate compressive force as the hinge or spring elements release the bending energy developed during expansion allowing the cardiac harness to follow the heart as it contracts and to apply contractile forces as well.

Other structural configurations for cardiac harnesses exist, however, but all have drawbacks and do not function optimally to treat CHF and other related diseases or failures. The present invention provides a novel approach to treat CHF and includes a cardiac harness that prevents electrical continuity circumferentially about the harness.

The present invention is directed to a cardiac harness that is fitted around at least a portion of the patient's heart to apply a compressive force on the heart during diastole and systole, and the harness includes a configuration that does not allow electrical continuity circumferentially about the harness. Therefore, if an electric current created by a defibrillation device (external defibrillator, automatic external defibrillator, or ICD) is applied to a patient who has a harness placed on their heart, the electric current will pass through the heart instead of being conducted around the heart through the harness. In one embodiment, the cardiac harness can be formed with a conductive material that is coated with a dielectric coating to prevent electrical continuity circumferentially about the harness. In another embodiment, various configurations of panels are connected together with a dielectric material, thereby electrically isolating each panel from one another in the harness. It is to be understood that several embodiments of cardiac wall tension reduction devices can be constructed and that such embodiments may have varying configuration, sizes, flexibilities, etc.

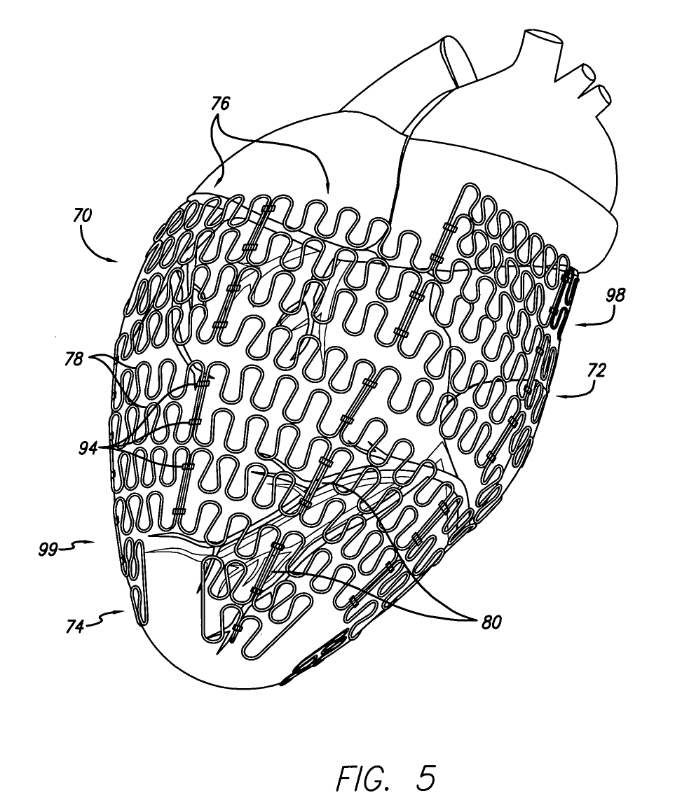

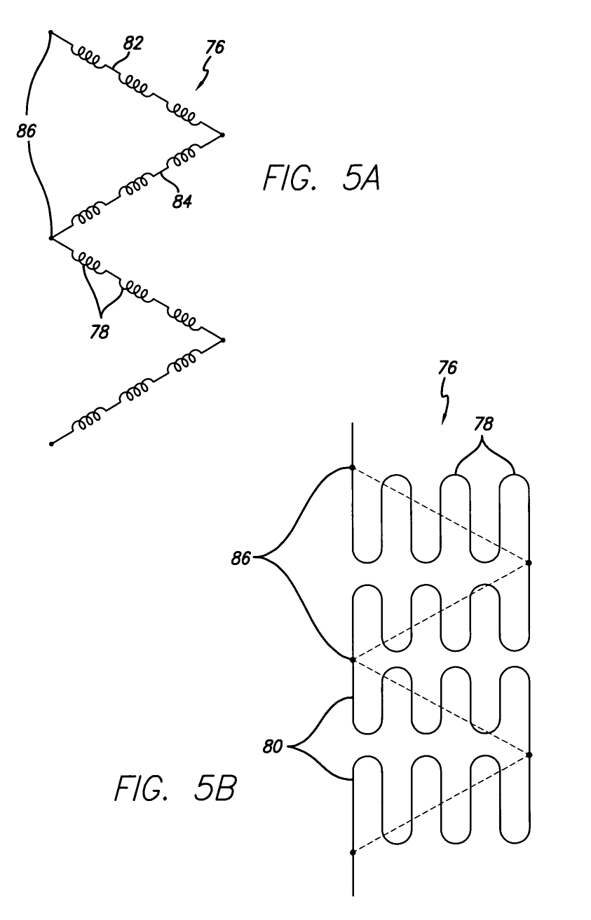

With next reference to FIG. 5, one embodiment of the present invention is shown of a cardiac harness 70 disposed upon a simulated heart 30. As shown, the harness extends longitudinally from a base portion 72 to an apex portion 74 of the heart. The harness comprises a plurality of spring arrays 76 that are interconnected with one another such that the harness circumferentially surrounds the heart. Each spring array comprises a plurality of spring elements 78 or members and a plurality of elongate portions 80. In the illustrated embodiment, the spring members are similar to the spring members discussed above with reference to FIGS. 2A and 2B. As will be discussed below, the elongate portions facilitate attaching each spring array to adjacent spring arrays. Each spring array preferably is formed of a metallic wire, preferably having a shape memory property, covered with a dielectric material.

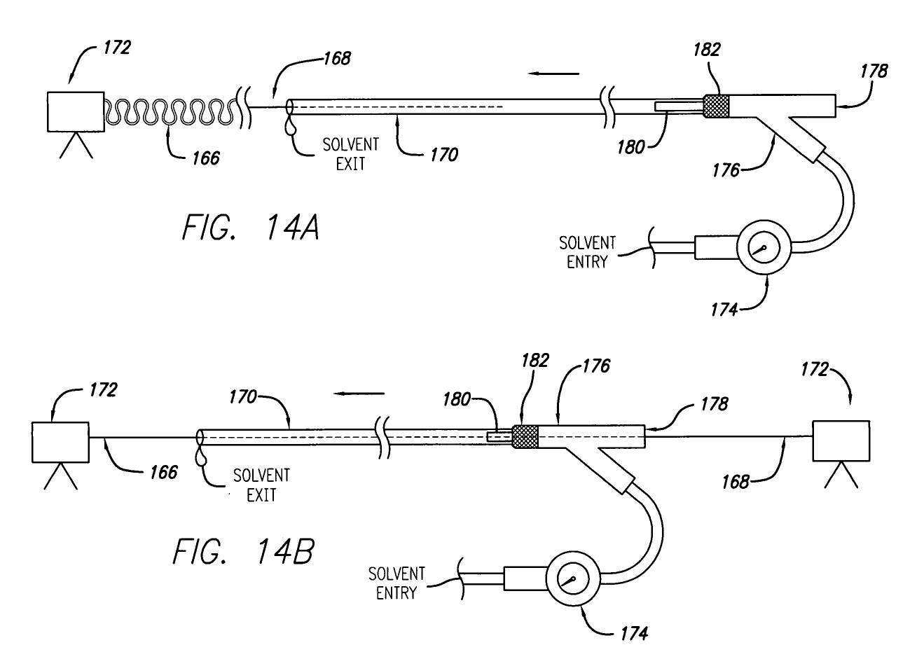

As discussed in the above-referenced applications, such wall tension reduction devices can be constructed from many suitable materials including various metals, fabrics, plastics and braided filaments. Suitable materials also include superelastic materials and materials that exhibit shape memory. More preferably, the spring arrays are formed of extruded Nitinol wire that is coated with silicone. Shape memory polymers can also be employed. Such shape memory polymers can include shape memory polyurethanes or other polymers such as those containing oligo(e-caprolactone) dimethacrylate and/or poly(ecaprolactone), which are available from mnemoScience. Methods of manufacturing the spring arrays are discussed below with reference to FIGS. 14A and 14B.

The spring arrays 76 provide a compressive force on the epicardial surface of the heart thereby relieving wall stress. In particular, the spring elements 78 expand and contract circumferentially as the heart expands and contracts during the diastolic and systolic functions. As the heart expands, the spring elements expand and resist expansion as they continue to open and store expansion forces. During systole, as the heart contracts, the spring elements will contract circumferentially by releasing the stored bending forces thereby assisting in both the diastolic and systolic function.

As just discussed, bending stresses are absorbed by the spring members during diastole and are stored in the members as bending energy. During systole, when the heart pumps, the heart muscles contract and the heart becomes smaller. Simultaneously, bending energy stored within the spring members is at least partially released, thereby providing an assist to the heart during systole. Although the harness is not intended to replace ventricular pumping, the harness does substantially assist the heart during systole.

With next reference to FIGS. 5A and 5B, each spring array 76 is generally “zigzag” shaped. As used herein, “zigzag” is a broad term and is used in its ordinary sense and refers, without limitation, to a series of turns, angles or alterations of course. FIG. 5A illustrates one embodiment of a spring array which serves as a conceptual model emphasizing the zigzag shape of the spring arrays of FIG. 5. As shown in FIG. 5A, the zigzag shape has a plurality of zig portions 82 interconnected with a plurality of zag portions 84. Each of the zig portions and zag portions have a plurality of interconnected spring elements 78. As used herein, “zig” is a broad term and is used in its ordinary sense and refers, without limitation, to one of the sections of a zigzag course which is typically at an angle relative to a zag, but may also be substantially parallel. Likewise, as used herein, “zag” is a broad term and is used in its ordinary sense and refers, without limitation, to one of the sections of a zigzag course which is typically at an angle relative to a zig, but may also be substantially parallel. Preferably, a zig is connected to a zag at their respective ends. In an additional embodiment, the ends of a zig and zag can be joined by a connector. As such, connected zigs and zags can be integrally formed or separately formed in alternative embodiments. Furthermore, the interconnections of the zig portions with the zag portions comprise a plurality of discrete locations 86 whereby the spring array 76 can be attached to other spring arrays. In the illustrated embodiment, the discrete locations are depicted as points of connection for a zig portion and a zag portion. It is to be understood that, in other embodiments, discrete locations can be elongate.

FIG. 5B illustrates a portion of one embodiment of a spring array 76. The dashed lines depict the general zigzag shape of the spring array. It will be appreciated that the spring array illustrated in FIG. 5B is substantially functionally similar to the spring array illustrated in FIG. 5A, but the spring array of FIG. 5B has a plurality of spring members 78 substantially similar to the spring members illustrated in FIG. 5. As further shown in FIG. 5B, the above-discussed discrete locations 86 are disposed in or on a plurality of elongate portions 80 interconnected with the spring members.

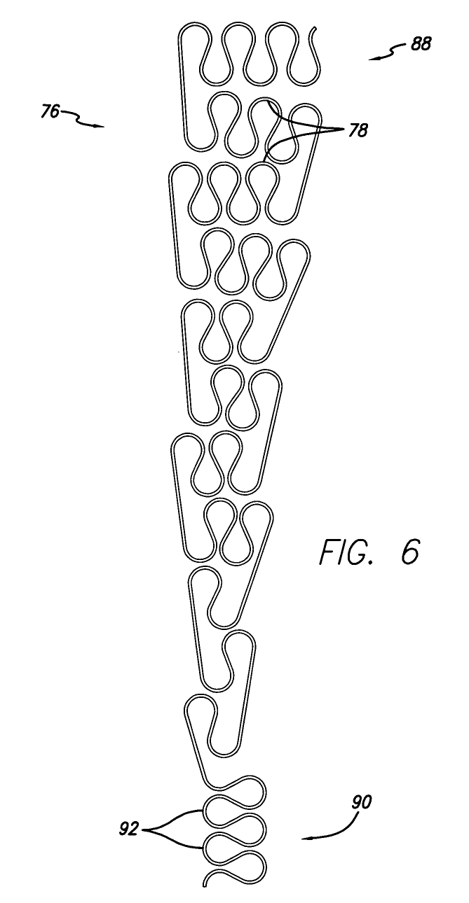

FIG. 6 illustrates a preferred embodiment of a spring array 76 for use in a cardiac harness. The spring array shown in FIG. 6 is similar to the spring arrays illustrated in FIGS. 5 and 5B. However, the spring array of FIG. 6 is shaped somewhat differently. For example, the FIG. 6 array has a decreasing number of spring members 78 in successive rows passing from a base end 88 to an apex end 90. This decrease in the number of spring members tapers the width of the spring array such that the assembled cardiac harness also tapers and thus better conforms to the general anatomy of the heart.

In the embodiment illustrated in FIG. 6, a plurality of longitudinal spring members 92 are included at the apex end 90 of the spring array 76. The longitudinal spring members facilitate attaching the apex end of the spring array to the ends of other spring arrays so as to enclose the cardiac harness around the apex portion of the heart. When the cardiac harness is installed on a heart, the longitudinal spring members from adjacent spring arrays may overlap one another. However, the longitudinal spring members remain very compliant. This arrangement allows significant motion of the apex of the heart in any direction with very little, if any, resistance from the harness. In order to help maintain the position of the harness on the heart, one or more stitches can optionally be applied to the heart at the apex in order to hold the overlapping spring members in place.

With reference again to the embodiment shown in FIG. 5, elongate portions 80 of each spring array 76 are attached to corresponding elongate portions of each adjacent spring array by a connector 94, preferably formed of a dielectric material 94, such as an adhesive, silicone, or other similar material. Preferably, the elongate portions are attached to one another such that there is a space between each pair of elongate portions, as shown in FIG. 5. The space, as well as the dielectric cover on the metallic wire, electrically isolates each spring array from the other spring arrays in the harness. This arrangement ensures that there is no electrical continuity circumferentially about the harness. Thus, if an electric current created by any defibrillator device (external defibrillator, automatic external defibrillator, ICD, etc.) is applied to a patient having a harness surrounding their heart, the electric current is not conducted around the heart through the harness, but instead passes through the heart unimpeded. As such, the effectiveness of the defibrillation is not defeated by the presence of the harness.

With continued reference to FIG. 5, in one embodiment a right side 98 of the cardiac harness 70 comprises longitudinally longer spring arrays 76 containing more rows of spring members than do the spring arrays on a left side 99 of the harness. As such, the harness extends higher on the right side of the heart than on the left side of the heart. Due to the anatomy of the human heart, the right atrium extends further from the apex of the heart than does the left atrium. The cardiac harness illustrated in FIG. 5 fits about the uppermost portion of the right atrium where the atrium begins to curve inwardly and also fits about the uppermost portion of the left atrium. As such, the harness fits better and is held more securely on the heart than if the right side of the harness were configured the same as the left side.

The harness 70 illustrated in FIG. 5 is configured so that no spring members 78 overlap one another. As such, wear of the harness due to repeated flexing and relative movement of the spring members is avoided.

As discussed briefly above, the cardiac harness 70 of FIG. 5 has a dielectric coating. The dielectric coating is configured to prevent an electric field created by a defibrillator device from being communicated by the metallic harness. As such, the electric field passes through the heart with little or no diminishment by the harness.

In this embodiment, the dielectric coating is silicone rubber. However, it is to be understood that various materials and methods can be used to coat the harness with dielectric material. For example, in one embodiment, an etched harness is coated with a layer of Parylene™, which is a dielectric polymer available from Union Carbide. Other acceptable materials include silicone rubbers and urethanes, as well as various polymers and the like. The materials can be applied to an etched harness by various methods, such as dip coating and spraying.

In accordance with one embodiment, a cardiac harness preferably is formed into a desired shape before being coated with dielectric material. For example, in one embodiment, Nitinol wire preferably is first treated and shaped to develop a shape memory of a desired spring member structure. Silicone tubing is then pulled over the wire. The wire then is returned to its shape memory shape. In another embodiment, Nitinol wire is dip coated with an insulating material.

In another embodiment, a harness is electrically insulated by stretching an extruded tube of flexible dielectric material over the harness. In a further embodiment, another flexible dielectric tube is disposed on the opposite side of the harness to effectively sandwich the harness between layers of flexible expandable dielectric material. Gaps may be formed through the dielectric material to help communicate the electric field through the harness to the heart. Specific methods for insulating the wire are discussed in more detail below with reference to FIGS. 14A and 14B.

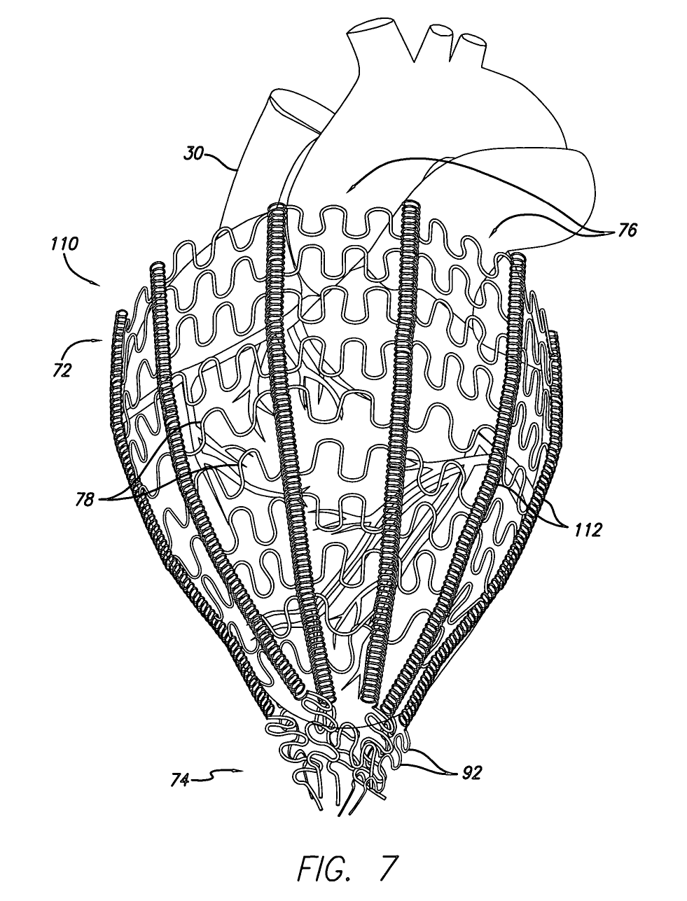

FIG. 7 illustrates another embodiment of a cardiac harness 110 for reducing cardiac wall tension. The cardiac harness is shown disposed upon a simulated heart 30. As shown, the harness extends longitudinally from a base portion 72 to an apex portion 74 of the heart. The harness has a plurality of spring arrays 76 that are attached to one another such that the harness circumferentially surrounds the heart. The spring arrays of the harness illustrated in FIG. 7 are substantially similar to the spring array illustrated in FIG. 6. As such, each spring array has a plurality of spring elements or members 78, a plurality of elongate portions (not shown in FIG. 7) and a plurality of longitudinal spring members 92. Furthermore, each spring array shown in FIG. 7 is generally zigzag shaped (FIGS. 5A-5B) and has a plurality of zig portions interconnected with a plurality of zag portions. The interconnections of the zig portions with the zag portions have a plurality of discrete locations whereby each spring array is attached to other spring arrays.

As shown in FIG. 7, each spring array 76 is attached to adjacent spring arrays by elongate coils 112. In the illustrated embodiment, each elongate coil is wound onto two adjacent spring arrays such that the respective elongate portions of the spring arrays are surrounded by windings of the coil. Because each spring array is coated with dielectric material, each spring array is electrically isolated from the other spring arrays in the harness and from the coils. This arrangement ensures that there is no electrical continuity circumferentially about the harness. Thus, if a defibrillation current provided by any defibrillation device is applied to a patient who has a harness placed on their heart, the defibrillation current is not conducted around the heart through the harness. Instead, the defibrillation current passes through the heart, and the effectiveness of the defibrillation is not defeated by the presence of the harness.

In accordance with yet another embodiment, selected ones of the elongate coils 112 of the cardiac harness 110 depicted in FIG. 7 are electrically connected to an electronic controller or the like. In this manner, the coils can be used as electrodes for a defibrillator, pacemaker or the like.

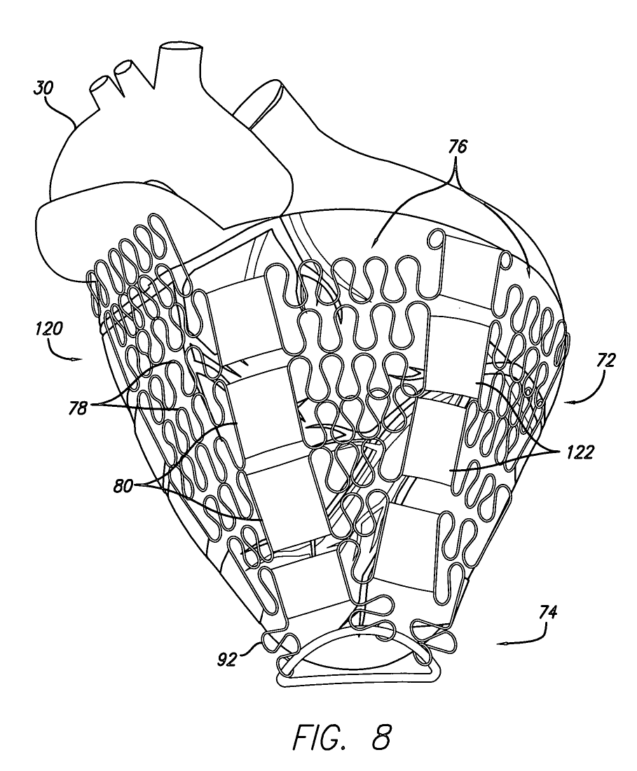

FIG. 8 illustrates another embodiment of a cardiac harness 120 disposed upon a simulated heart 30. As shown, the harness extends longitudinally from a base portion 72 to an apex portion 74 of the heart. The harness has a plurality of spring arrays 76 that are attached to one another such that the harness circumferentially surrounds the heart. The spring arrays illustrated in FIG. 8 have a zigzag structure similar to the spring arrays illustrated in FIGS. 5 and 6. Thus, each spring array has a plurality of spring elements or members 78, a plurality of elongate portions 80 and a plurality of longitudinal spring members 92.

As shown in FIG. 8, each spring array 76 is attached to adjacent spring arrays by a plurality of nonconductive connectors 122. In the illustrated embodiment, each nonconductive connector has a layer of polymer that is connected to the elongate portions of adjacent spring arrays by adhesive or other mode of connection. Preferably, each connector is generally inelastic or has relatively low elasticity so that the elastic expansion and contraction of the harness is generally controlled by the properties of the spring arrays. It is contemplated that the nonconductive connectors may include any medical grade polymer such as, but not limited to, polyethylene, polypropylene, polyurethane, nylon, PTFE and ePTFE. Of course, in additional embodiments, at least some of the connectors can be formed of an elastic material, such as silicone rubber, that contributes to the mild circumferential force applied to the heart by the harness.

With continued reference to FIG. 8, the nonconductive connectors 122 define a space between adjacent spring arrays 76. As such, each spring array is electrically isolated from the other spring arrays in the harness by the space. This arrangement provides no electrical continuity circumferentially about the harness. Thus, if a an electric current created by a defibrillation device is applied to a patient who has a harness that is placed on their heart, the electric current is not conducted around the heart through the harness. Instead, the electric current passes through the heart unimpeded, and the effectiveness of the defibrillation is not defeated by the presence of the harness.

In the harness embodiment illustrated in FIG. 8, the spring arrays 76 are not coated with a dielectric material. It is to be understood, however, that some or all of the spring arrays may be coated with a dielectric in order to further diminish any electrical continuity around the heart.

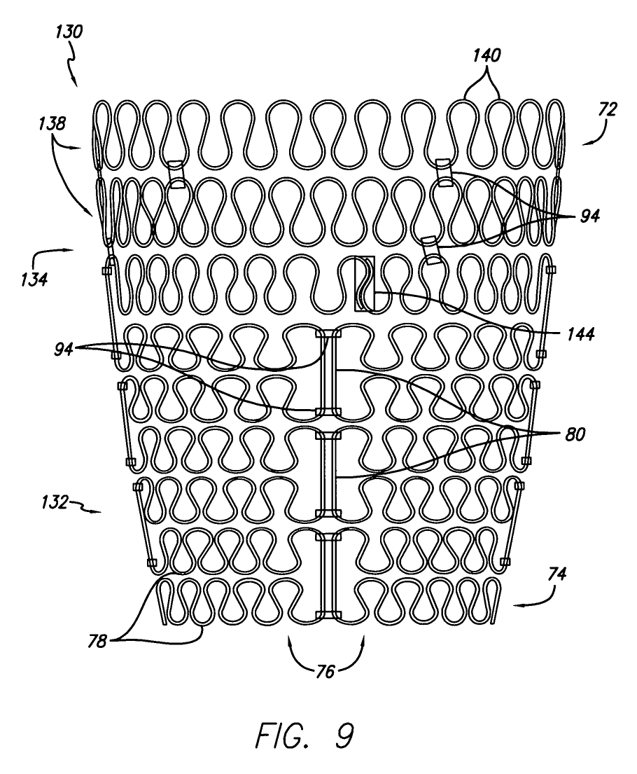

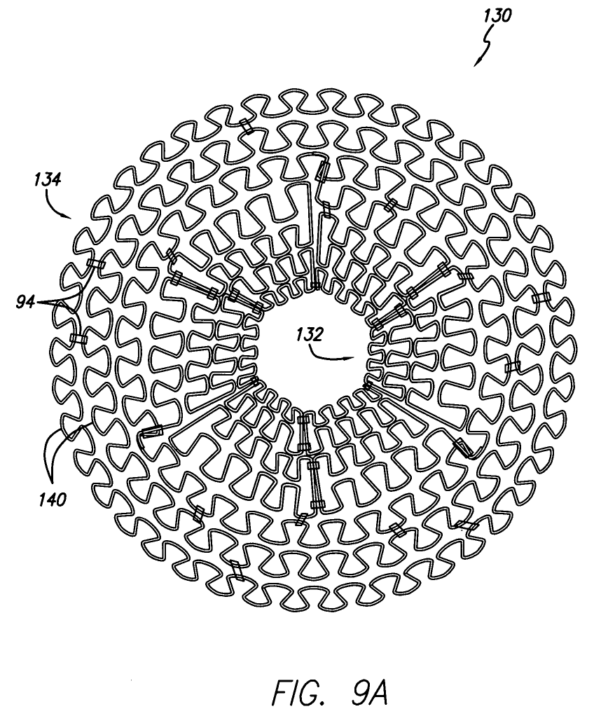

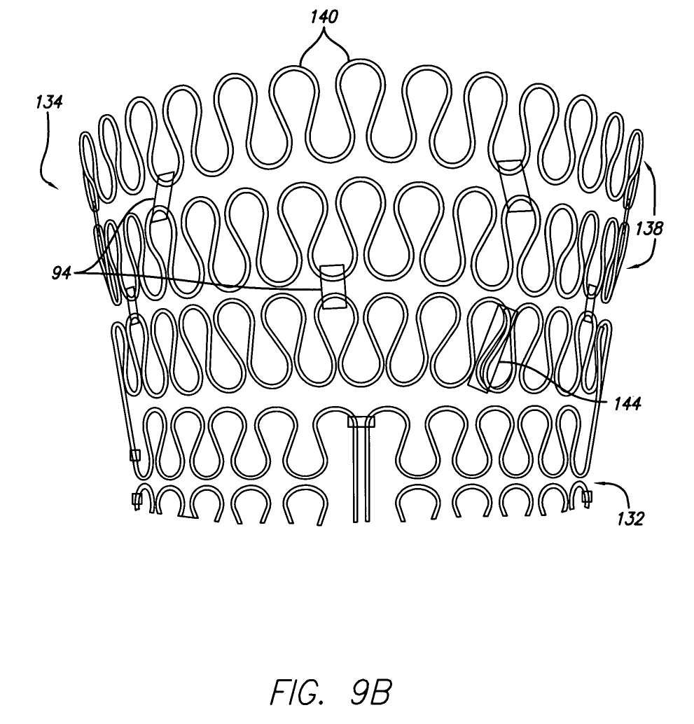

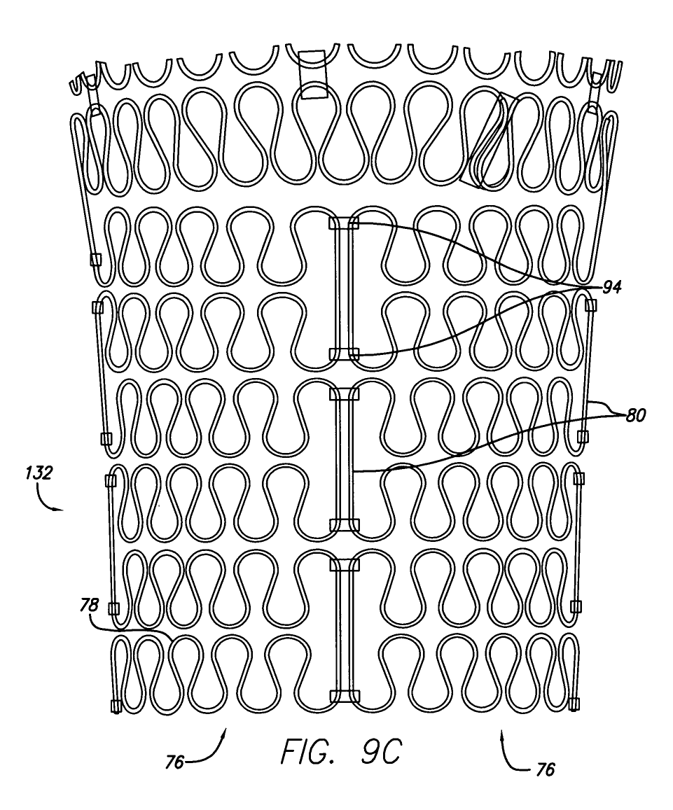

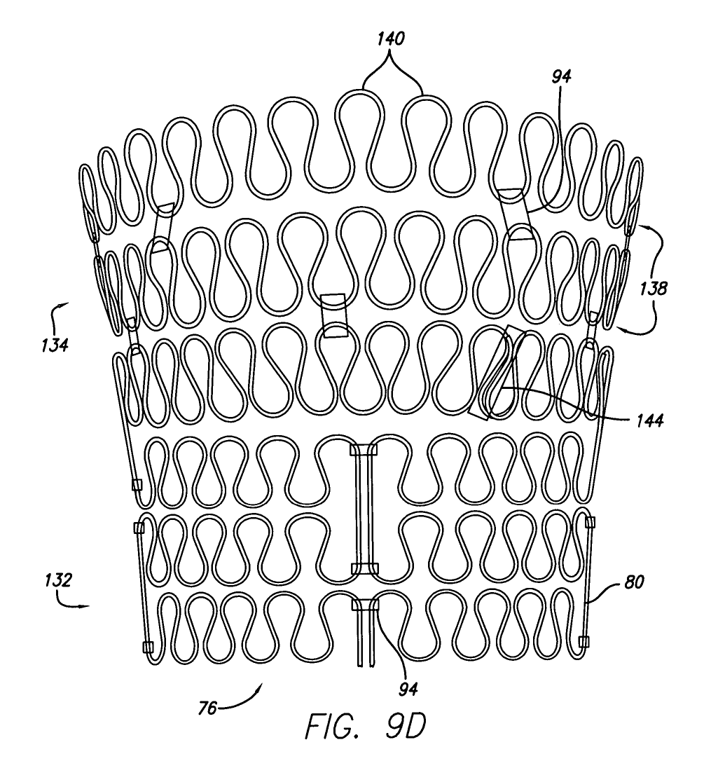

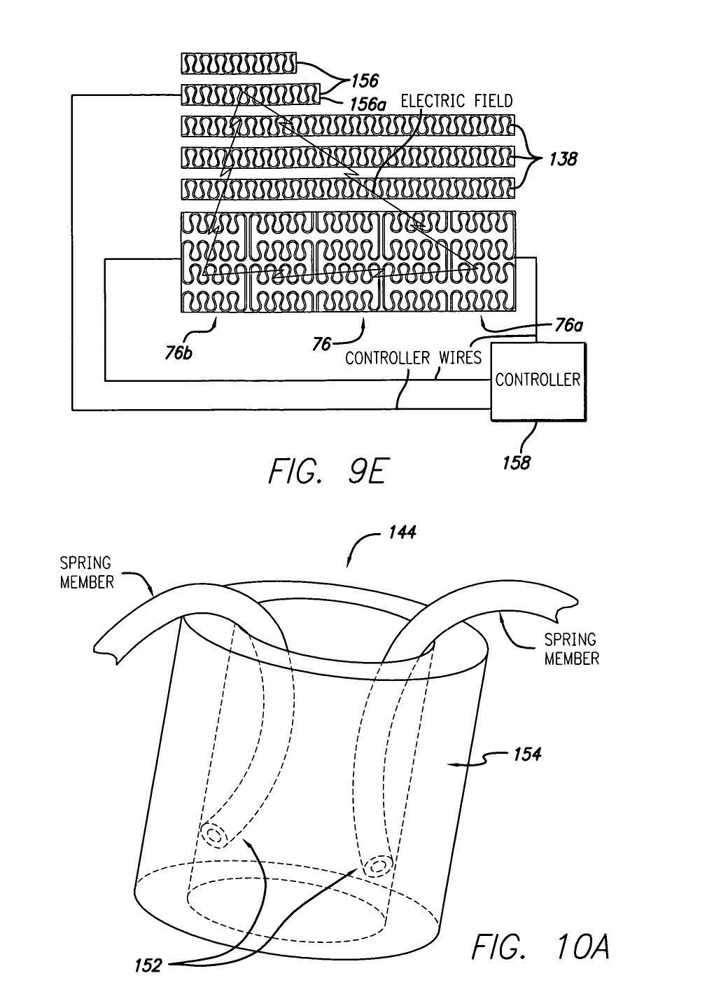

FIGS. 9 through 9D illustrate another embodiment of a cardiac harness 130 for reducing cardiac wall tension. The cardiac harness is configured to circumferentially surround a patient's heart and extends longitudinally from a base end 72 to an apex end 74. The harness of FIG. 9 has a first portion 132 and a second portion 134. The first portion is configured to be disposed closer to the apex portion of the heart than the second portion.

As best shown in FIG. 9C, the first portion 132 has a plurality of spring arrays 76. The spring arrays illustrated in FIG. 9C share similarities with the spring arrays illustrated in FIGS. 5 and 6, in that each spring array in the first portion has a plurality of spring members 78 interconnected with a plurality of elongate portions 80. Furthermore, each spring array shown in FIG. 9C is generally zigzag shaped and has a plurality of zig portions interconnected with a plurality of zag portions.

As shown in FIG. 9C, the elongate portions 80 of each spring array 160 are attached to corresponding elongate portions of adjacent spring arrays by a nonconductive bond or connector 94, such as an adhesive, silicone rubber or other similar material. In the illustrated embodiment, the elongate portions are attached to one another such that a dielectric layer is disposed between each pair of elongate portions. Also, each array is coated with a dielectric coating. As such, each spring array is electrically isolated from the other spring arrays in the harness, and there is no electrical continuity circumferentially about the first portion of the harness.

As best shown in FIG. 9B, the second portion 134 of the cardiac harness 130 has a plurality of circumferentially extending rings 138 disposed longitudinally adjacent to one another. Each ring has a plurality of interconnected spring elements or members 140 covered with a dielectric material. The spring members shown in FIGS. 9 through 9D are similar to the spring members discussed above with reference to FIGS. 2A and 2B. A plurality of nonconductive connectors 94 interconnects adjacent rings, and also interconnects the second portion and first portion of the harness. Preferably, the nonconductive connectors have a semi-compliant design, and are formed of silicone rubber or other similar material. More preferably, the connectors are formed of the same material used for the dielectric coating.

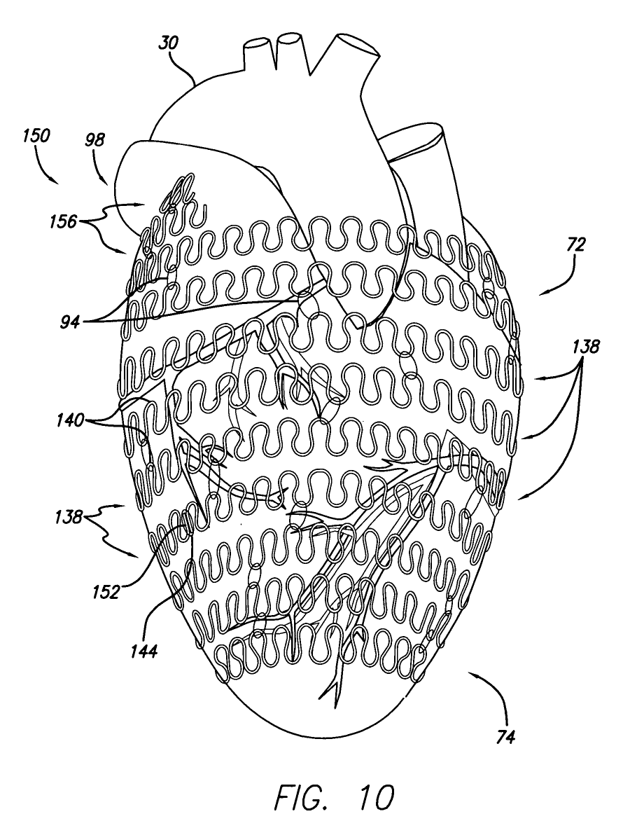

In one embodiment, each ring 138 is formed by first forming an elongate series of spring members 142, as shown in FIG. 11, and then joining the opposite ends of the series together to form the ring-shaped configuration shown in FIG. 9B. It will be appreciated that the lengths of the elongate series are selected such that the resulting rings are sized in conformity with the general anatomy of the patient's heart. More specifically, some rings have more spring members than others. With reference again to FIGS. 9B and 9D, the opposite ends of each circumferentially extending ring are attached to one another by a connective junction 144.

In the embodiment illustrated in FIGS. 9-9D, the rings 138 in the second portion 134 are configured to be circumferentially stiffer than the attached arrays 76 in the first portion 132. As such, the harness has a firmer grip about the base portion of the patient's heart. This arrangement helps anchor the harness securely onto the heart.

It will be appreciated that because the rings 138 are coated with dielectric material and are interconnected by a nonconductive material, each ring is electrically insulated from the other rings in the second portion 134 of the harness 130, as well as from the first portion 132 of the harness. Furthermore, the dielectric material ensures that there is no electrical continuity across the connective junctions 144, and that there is no electrical continuity either circumferentially about the harness or longitudinally across the harness. As such, when an electric current is created using a defibrillation device, the electric current is not conducted around the heart through the harness, but rather travels through the heart unimpeded. Thus, the effectiveness of the defibrillation is not defeated by the presence of the harness.

In a still further embodiment, two or more of the spring arrays 76 in the first portion 132 are not coated with a dielectric, but are connected to a controller configured to selectively electrically charge the array. In this manner, the arrays function as electrodes for a defibrillator, pacemaker or the like. At least one pair of the non-coated electrode-arrays preferably are insulated from one another by a dielectric-covered array. As such, current flowing between the electrode-arrays is forced to flow through the heart rather than through the rest of the harness. In this design there is also no shunting or shielding of the electrical current through the cardiac harness, and the cardiac harness does not develop into a Faraday cage.

With next reference to FIG. 10, another embodiment of a cardiac harness 150 is illustrated disposed on a simulated heart 30. As shown, the cardiac harness is configured to circumferentially surround the heart and extend longitudinally from a base portion 72 to an apex portion 74 of the heart. The harness has a plurality of circumferentially extending rings 138 disposed longitudinally adjacent to one another. Each ring has a plurality of interconnected spring members 140 covered with a dielectric material. The spring members shown in FIG. 10 are similar to the spring members discussed above with reference to FIGS. 2A and 2B. A plurality of nonconductive connectors 94 interconnects adjacent rings. The nonconductive connectors have a length oriented longitudinally relative to the rings so as to create space between adjacent rings. Preferably, the nonconductive connectors are formed of a semi-compliant design using silicone rubber or other similar material.

In one embodiment, each ring initially has an elongate strand or series of spring elements 142, as shown in FIG. 11. Each elongate strand has a series of the above-discussed spring members 140. During manufacturing of the cardiac harness, each elongate strand is cut to a length such that when opposite ends of the elongate strand are bonded together, the elongate strand assumes the ring-shaped configuration shown in FIG. 10. It will be appreciated that the lengths of the elongate strands are selected such that the resulting rings 138 are sized in conformity with the general anatomy of the patient's heart. More specifically, strands in the apex portion 74 of the harness are not as long as the strands used to form the base portion 72. As such, the harness generally tapers from the base toward the apex in order to generally follow the shape of the patient's heart. In another embodiment, the diameter of a ring at the base of the harness is smaller than the diameter of the adjacent ring. In this embodiment, the harness has a greatest diameter at a point between the base and apex ends, and tapers from that point to both the base and apex ends. Preferably, the point of greatest diameter is closer to the base end than to the apex end. It is contemplated that the lengths of the strands, as well as the sizes of the spring members, may be selected according to the intended size of the cardiac harness and/or the amount of compressive force the harness is intended to impart to the patient's heart.

As shown in FIG. 10, opposite ends 152 of each circumferentially extending ring 138 are attached to one another by a connective junction 144. In one embodiment, illustrated in FIG. 10A, each connective junction has a small tube segment 154 into which the opposite ends of the ring are inserted. The tube segment serves to prevent the opposite ends of the ring from tearing loose from one another after the harness is placed on the heart. Preferably, each tube segment is filled with a dielectric material such as silicone, or other similar material after the ring-ends are placed therein.

With continued reference to FIG. 10, the right side 98 of the base portion 72 of the harness 150 has partial strands 156 of interconnected spring members 140. Preferably, the partial strands are connected to the adjacent full ring 138 in a manner so that the partial strands are stretched. As such, the partial strands will bend inwardly to “cup” the upper portion of the right atrium, as simulated in FIG. 10.

It will be appreciated that because the rings are coated with dielectric material and are interconnected by nonconductive material, each ring is electrically isolated from the other rings in the harness. As such, there is no electrical continuity either circumferentially about the harness or longitudinally along the harness. Thus, if an electric current created by a defibrillation device is applied to a patient who has a harness placed on their heart, the electric current passes through the heart rather than being conducted around the heart through the harness. As a result, the effectiveness of the defibrillation is not defeated by the presence of the harness.

In a still further embodiment, the harness 130 depicted in FIGS. 9-9D includes at least one partial strand 156 as discussed above with reference to FIG. 10. With reference next to FIG. 9E, an embodiment is illustrated wherein one of the partial strands 156a is not covered with a dielectric material, but is electrically connected to a controller 158 and, similarly, two of the spring arrays 76a and 76b are connected to the controller and are not electrically insulated from the heart. As such, the non-coated partial strand and spring arrays (electrode members) function as electrodes for a defibrillator or pacemaker. In accordance with one embodiment, the controller energizes the electrode members in a manner to create a generally triangular electric field between the electrodes so as to enable defibrillation.

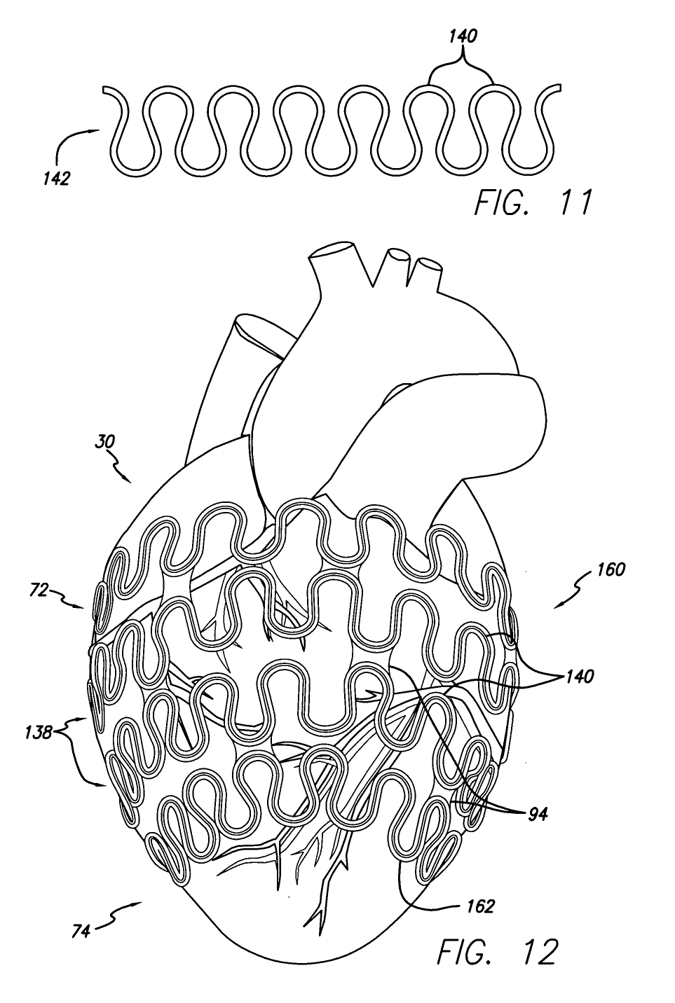

FIG. 12 illustrates another embodiment of a cardiac harness 160 disposed on a simulated heart 30. As shown, the cardiac harness is configured to circumferentially surround the heart and extends longitudinally from a base portion 72 to an apex portion 74 of the heart. The harness has a plurality of circumferentially extending rings 138. Each ring includes a plurality of interconnected spring members 140 covered with a dielectric material 162. The spring members shown in FIG. 12 are similar to the spring members discussed above with reference to FIGS. 2A and 2B. A plurality of nonconductive connectors 94 interconnects adjacent rings. In one embodiment, the nonconductive connectors are formed of the dielectric material which covers the spring members.

In one embodiment, each ring 138 initially has an elongate strand 142, as discussed above with reference to FIG. 11. Each elongate strand includes a series of the above-discussed spring members 140. During manufacturing of the cardiac harness, each elongate strand is cut to a length such that when opposite ends of the elongate strand are secured together, the elongate strand assumes the ring-shaped configuration shown in FIG. 12. The lengths of the elongate strands are selected such that the resulting rings are sized in conformity with the general anatomy of the patient's heart. In one embodiment, the lengths of the strands, as well as the sizes of the spring members, are selected according to the intended size of the cardiac harness and/or the amount of compressive force the harness is intended to impart to the patient's heart.

Once the elongate strands are formed into the ring-shaped configuration shown in FIG. 12, the rings 138 are covered with a dielectric material. The dielectric covering is configured to prevent an electric current applied by a defibrillation device from being communicated by the rings of the harness. As such, the electric current passes through the heart with little or no diminishment by the harness.

Various materials and methods can be used to coat the harness with dielectric material. In the illustrated embodiment, the rings are coated with silicone rubber. Other acceptable materials include Parylene™ and urethanes, as well as various polymers and the like. The materials can be applied to a harness by various methods, such as dip coating and spraying.

In the illustrated embodiment, the rings are placed on a mandrel and coated with dielectric material. It is contemplated that such a mandrel has an exterior surface configured such that the resulting ring structure is sized in conformity with the general anatomy of a human heart. Excess dielectric material is then removed from the cardiac harness, such that the shape of the dielectric material generally follows the shape of the spring members 140, as shown in FIG. 12. In this embodiment, excess dielectric material is left intact between some of the spring members of adjacent rings so as to provide nonconductive connectors 94 between adjacent rings. The excess dielectric material may be removed from the harness by using any cutting tool, such as a scalpel, laser, water jet, or the like.

As shown in FIG. 12A, a connective junction 164 joins opposite ends 152 of each circumferentially extending ring 138. In one embodiment, the material including the dielectric sheet secures the opposite ends of the rings. In another embodiment, the opposite ends of each ring may be further secured by applying silicone, or another similar material, before the dielectric material is applied to the harness. Also, the opposing ends may be welded, soldered, adhesively bonded, or held together by other means. In still another embodiment, the connective junctions may each have a small tube segment into which the opposite ends of the ring are inserted prior to application of the dielectric sheet to the harness. As discussed with reference to FIG. 10A, the tube segment serves to prevent the opposite ends of the ring from tearing loose after the harness is placed on the heart. In this embodiment, the tube segments are covered with the dielectric material including the sheet of dielectric material.

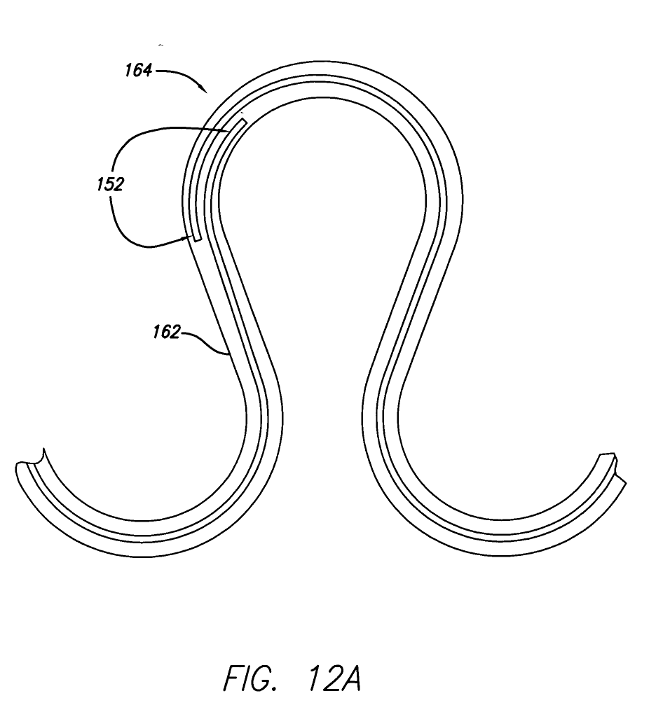

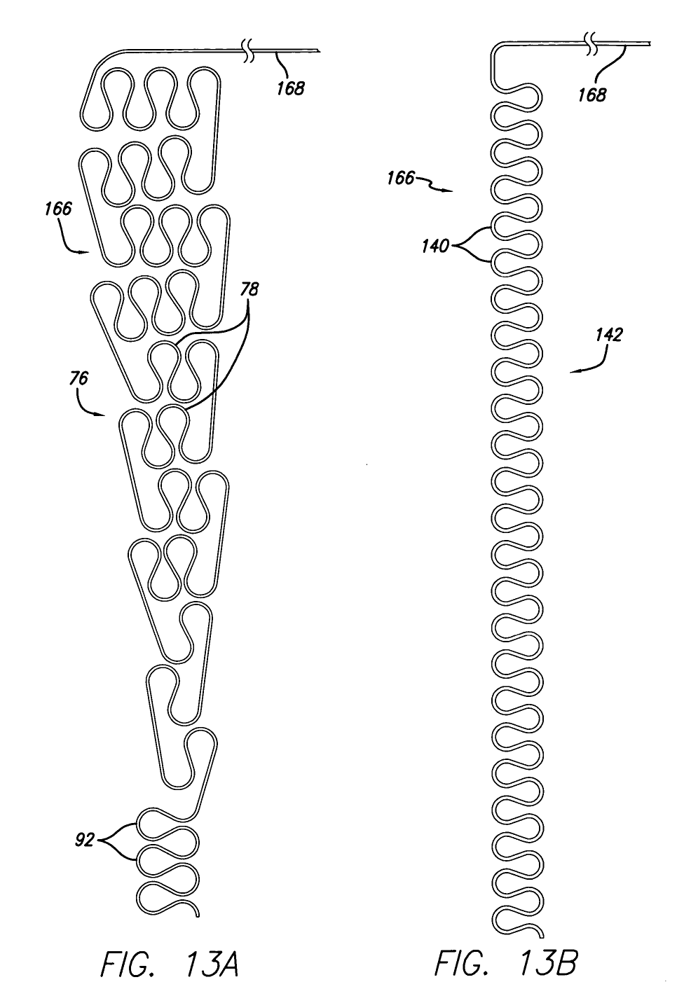

A method of manufacturing a cardiac harness is now described with reference to FIGS. 13A through 14B. The method generally includes configuring a metallic wire, and then covering the wire with an electrically insulative material. In one embodiment, Nitinol wire is first treated and shaped to develop a “remembered” shape having a harness portion 166 and a leader portion 168, as shown in FIGS. 13A and 13B. The harness portion has a plurality of spring members that are preferably arranged into a predefined configuration, such as a spring array 76 illustrated in FIG. 13A or an elongate strand 142 shown in FIG. 13B. While held in the predefined configuration, the harness portion preferably is heat-set at a temperature of about 520° C. for about 20 minutes to establish the shape memory. The wire is then electropolished in accordance with standard methods known in the art. As shown in FIGS. 13A and 13B, the wire is configured such that the leader portion is disposed at one end of the harness portion of the wire.

Once the harness portion 166 of the wire is configured as described above, the wire is then covered with an electrically insulative material. In one embodiment, a tube of dielectric material is pulled over the wire. In a preferred embodiment, the tube is formed of silicone rubber. It will be appreciated that the inner diameter of the tube determines the level of tightness between the tube and wire. In one embodiment, wherein the wire has a diameter of about 0.012 inches, a silicone tube having an inner diameter of about 0.012 inches provides a relatively tight fit. In another embodiment, wherein the wire has a diameter of about 0.012 inches, a silicone tube having an inner diameter of about 0.020 inches provides a relatively loose fit. A silicone tube having an inner diameter smaller than the diameter of the wire can also be used to obtain a snug fit. In a preferred embodiment, silicone tubing sold under the trademark Nusil MED 4755 is used.

FIGS. 14A and 14B illustrate an apparatus and method for drawing a silicone rubber tube 170 over a harness portion 166, such as the portions illustrated in FIGS. 13A and 13B. The apparatus includes a clamp 172 into which one end of the harness portion is clamped. The leader portion 168 of the harness portion is preferably free. A pressure source 174 supplies solvent under a substantially constant pressure of, preferably, less than about 5 atmospheres and, more preferably, between about 1 to 2 atmospheres. The pressure source applies the solvent to a connector 176 which comprises a Y-shaped adapter. The solvent is supplied to one of the Y branches, another of the Y branches includes a compression valve 178, such as a Touhy-borst valve. Preferably, a hollow needle 180 extends from a base portion 182 of the Y adapter.

With particular reference to FIG. 14A, the silicone tube 170 preferably is threaded over the outer diameter of the hollow needle 180. As such, solvent is supplied through the needle to the tube. In one embodiment, the solvent primarily includes a lubricant which facilitates sliding the tube over wire. Preferably, the lubricant is comprised of DOW OS-10, isopropyl alcohol (IPA), or another similar substance. In another embodiment, the solvent is a substance which primarily swells the inner diameter of the tube so as to facilitate sliding the tube over the wire. In such an embodiment, the solvent preferably includes hexane, heptane, xylene, and the like.

With continued reference to FIG. 14A, once the solvent is flowing within and through the silicone tube, the free end of the leader portion 168 is threaded into the tube and the tube is advanced over the leader until the entire tube is disposed on the leader portion. With reference next to FIG. 14B, once the leader portion has been advanced completely through the tube, the leader portion is threaded through the hollow needle and through the Touhy-borst valve 178 of the Y adapter. The free end of the leader portion is then clamped in place.

Due to the tortuous path defined by the spring elements 78 or 140, it may be difficult for the tubing 170 to be slid over the harness portion 166 without deforming the spring elements. However, in accordance with one embodiment, and with the assistance of the solvent, the tubing is drawn over the harness portion taking care not to substantially stretch the spring members. In accordance with another embodiment, the wire is pulled straight and held tightly in place between the clamps. In this manner, it is quite easy to advance the tubing over the harness portion because the spring elements of the harness portion have been substantially straightened out, as illustrated in FIG. 14B. Once the tubing is disposed completely over the harness portion, the clamps 172 are released and, due to the shape memory and superelastic properties of Nitinol, the harness portion springs back substantially to its shape memory configuration. In accordance with a still further embodiment, once the free end of the leader has been clamped, the entire wire is stretched so that the spring elements of the harness portion are partially deformed, but are not stretched straight. In this manner, it becomes relatively easy to slide the tubing over the spring elements of the harness portion, but the spring elements are not deformed so much as to compromise their preformed memory shape. In this embodiment, care is taken to further deform the spring elements as little as possible while sliding the tubing into place.

In each of these embodiments, once the tube 170 has reached the end of the wire, and thus is covering the entire harness portion 166, the supply of pressurized solvent is stopped and the solvent supply apparatus is removed. The ends of the wire are removed from the clamps 172 and the leader portion 168 is trimmed from the harness portion. The harness portion substantially assumes its shape memory shape, and is ready to be further formed into a cardiac harness.

In order to relieve localized stresses that may exist between the tubing 170 and the wire, the tubing/wire combination is exposed to low level vibrations in order to help the tubing relax and shrink to a relaxed condition on the wire. In a preferred embodiment, the tubing/wire combination is treated with an ultrasonic cleaner which ultrasonically vibrates the combination. Such vibration can be termed “micromotion”, and helps the tubing and wire achieve a state of equilibrium relative to one another. As such, localized stresses that may have formed as the tubing was advanced over the wire are relaxed.

Although the present invention has been described in terms of certain preferred embodiments, other embodiments that are apparent to those of ordinary skill in the art are also within the scope of the invention. Accordingly, the scope of the invention is intended to be defined only by reference to the appended claims. While the dimensions, types of materials and coatings described herein are intended to define the parameters of the invention, they are by no means limiting and are exemplary embodiments.

We claim:

1. A cardiac harness configured to fit about a patient's heart, the harness comprising a conductive material, the conductive material being coated with a dielectric coating to electrically insulate at least the heart tissue from the conductive material.

2. The cardiac harness of claim 1, wherein the conductive material is entirely coated with the dielectric coating so that the entire harness is electrically insulated.

3. The cardiac harness of claim 1, wherein the harness is coated with a layer of Parylene™ about 5 microns thick.

4. The cardiac harness of claim 1, wherein the harness is coated with silicone rubber.

5. The cardiac harness of claim 1, wherein the dielectric coating comprises an elastomer.

6. The cardiac harness of claim 1, wherein the dielectric coating includes urethane.

7. The cardiac harness of claim 1, wherein the dielectric coating includes polytetrafluoroethylene.

8. The cardiac harness of claim 1, wherein the dielectric coating includes Parylene™.

9. The cardiac harness of claim 1, wherein the conductive material comprises a wire formed into a plurality of hinge members.

10. A method of manufacturing a cardiac harness, comprising:
providing a metallic wire;
forming the wire into a plurality of spring members; and
covering the wire with a dielectric material.

11. The method of claim 10, wherein the covering comprises:
introducing a fluid into a tube; and
sliding the tube over the wire.

12. The method of claim 11, wherein a solvent is introduced into the tube.

13. The method of claim 11, wherein the sliding comprises sliding the tube onto a leader portion of the wire and sliding the tube from the leader portion onto a harness portion of the wire comprised of the spring members arranged in a first configuration.

14. The method of claim 13, wherein the sliding onto the harness portion comprises changing the shape of the spring members by straightening the harness portion of the wire, the method further comprising substantially returning the shape of the spring members to substantially the first configuration.

15. The method of claim 10, wherein the dielectric material comprises silicone.

16. The method of claim 10, wherein the wire is formed into the spring members prior to being covered with the dielectric material.

17. The method of claim 16, wherein the covering comprises:
applying the dielectric material to the wire such that the wire is insulated by the dielectric material; and
removing excess dielectric material from the wire so that the shape of the dielectric material generally follows the shape of the spring members.

18. The method of claim 17, wherein the removing comprises laser cutting the dielectric material.

19. The method of claim 17, wherein the dielectric material comprises silicone.

20. The method of claim 10, wherein the wire is coated with the dielectric material prior to being formed into a plurality of spring members.

21. A method of manufacturing a cardiac harness, comprising:
providing a flat sheet of conductive material;
etching at least one spring member out of the conductive material; and
coating the etched spring member with a dielectric material.

22. The method of claim 21, wherein the coating comprises:
applying the dielectric material to the etched spring member so that the etched spring member is insulated by the dielectric material; and
removing excess dielectric material from the etched spring member so that the shape of the dielectric material generally follows the shape of the spring members.

23. The method of claim 22, wherein the removing comprises laser cutting the dielectric material.

24. The method of claim 22, wherein the dielectric material comprises silicone.

25. A cardiac harness which circumferentially surrounds a patient's heart and extends longitudinally from an apex portion to a base portion of the heart, comprising:
a first portion and a second portion, the first portion configured to be disposed closer to an apex portion of the heart than the second portion;
the first portion comprising a plurality of interconnected panels that are electrically insulated from one another along respective longitudinal sides to inhibit electrical conduction circumferentially about the harness; and
the second portion being electrically insulated from the first portion.

26. The cardiac harness of claim 25, wherein the second portion comprises a plurality of circumferentially extending rings comprising a plurality of interconnected spring elements, the rings being electrically insulated from one another.

27. The cardiac harness of claim 25, wherein the first portion is connected to the second portion by at least one non-conductive connector.

28. The cardiac harness of claim 27, wherein the first and second portions are coated with a dielectric material, and the at least one non-conductive connector comprises the dielectric material.

29. A cardiac harness, comprising:
a first spring array and a second spring array, each spring array comprising a plurality of zig portions interconnected with a plurality of zag portions such that the array is generally zigzag shaped, each of the zig portions and zag portions comprising a plurality of interconnected spring elements;
wherein the first and second spring arrays are connected to one another at a plurality of discrete locations corresponding to interconnections of a zig portion with a zag portion.

30. The cardiac harness of claim 29, additionally comprising an elongate coil, one or more windings of the coil surrounding portions of adjacent spring arrays at at least some of the discrete locations.

31. The cardiac harness of claim 29, wherein the spring array is formed from a single piece of material.

32. The cardiac harness of claim 29, wherein the spring array is formed from shape memory material.

33. A cardiac harness configured to fit about a patient's heart, the harness comprising a plurality of interconnected spring members comprised of a conductive material, at least some of the spring members connected to other spring members by a dielectric material such that the dielectric connected spring members are substantially electrically insulated from each other.
